(19) United States
(12) Patent Application Publication
(10) Pub. No.: US 2014/0357962 A1
(43) Pub. Date: Dec. 4, 2014

(54) OBJECTIVE NON-INVASIVE METHOD FOR QUANTIFYING DEGREE OF ITCH USING PSYCHOPHYSIOLOGICAL MEASURES

(71) Applicant: The Procter & Gamble Company, Cincinnati, OH (US)

(72) Inventors: Nick Robert HARRINGTON, Boston, MA (US); James Robert SCHWARTZ, West Chester, OH (US); James Charles DUNBAR, Morrow, OH (US); Haruko MIZOGUCHI, Fairfield, OH (US)

(73) Assignee: The Procter & Gamble Company, Cincinnati, OH (US)

(21) Appl. No.: 14/289,589

(22) Filed: May 28, 2014

Related U.S. Application Data
(60) Provisional application No. 61/827,928, filed on May 28, 2013.

(51) Int. Cl. A61B 5/00; A61B 5/0205

(52) U.S. Cl. CPC A61B 5/445; A61B 5/0205; A61B 5/4848; A61B 5/6826; A61B 5/6829; A61B 5/0533
USPC 600/301; 600/558; 600/481; 600/529; 600/306; 600/508

(57) ABSTRACT

An objective non-invasive method for quantifying itch comprising the steps of presenting a visual stimulus to a subject, wherein the visual stimulus comprises focusing the subject on itch; collecting psychophysiological data from subject while presenting the visual stimulus; using the psychophysiological data to objectively assess the therapeutic value of a treatment or a product.

### FIELD OF THE INVENTION

The present invention is directed to an objective non-invasive method for quantifying itch using psychophysiological measures.

### BACKGROUND OF THE INVENTION

Historically, measuring scalp irritation has relied on personal subjective ratings; these measures, however, are subject to conscious and unconscious bias (e.g., time of day & day of week, temperature, competing stimuli, time since last washed hair, difficulty in describing an abstract concept such as scalp itch) and can result in highly variable data. Reduction in scalp irritation is a big unmet subject need. The present invention has demonstrated that novel behavioral science techniques can provide a viable objective method of a subjective subject experience.

The importance of the symptom of itch in scalp dermatitis has been evaluated in the past. The most frequently described condition associated with unhealthy scalp is itch. This is also the most bothersome symptom, having the largest negative influence on sufferers' quality of life.

With regard to the general physiology of itch, the perception of itch represents the end of a complex physiological pathway that is initiated at the skin surface. A number of stimuli can start the cascade of events that eventually lead to the perception of itch.

Objectively measuring feelings such as pain, itchiness and irritation is an important unmet need in order to create products to reduce negative consumer experiences (e.g., reduction in scalp itch associated with dandruff). Such feelings, however, are abstract and the ability to describe or rate, them varies by an individual's sensitivity and their ability to articulate the experience.

Non-invasive psychophysiological measures (e.g., measures of electrodermal activity, heart rate and respiration) reflect emotional reactivity and have the potential to provide an objective measure of subject's response / engagement to both positive and negative consumer experiences.

In the present invention, specifically, when itchy scalp sufferers are presented with itch stimuli they show an increase in electrodermal activity, a decrease in respiration (analogous to holding your breath after experiencing a startling event) and a reduction in heart rate.

### SUMMARY OF THE INVENTION

An embodiment of the present invention is directed to an objective non-invasive method for quantifying itch comprising the steps of presenting a visual stimulus to a subject, wherein the visual stimulus comprises focusing the subject on itch; collecting psychophysiological data from subject while presenting the visual stimulus; using the psychophysiological data to objectively assess the therapeutic value of a treatment or a product.

### BRIEF DESCRIPTION OF THE DRAWINGS

FIG. 1 is a diagram illustrating skin conductor sensor placement at the volar surface of the medial or distal phalanges of the fingers.

FIG. 2 is a series of graphs summarizing the differences in psychophysiological response of (A) elctrodermal activity (EDA), (B) heart rate, and (c) respiration rate between two groups of individuals self-assessed as itch and non-itch.

FIG. 3 compares the therapeutic impact, as measured by EDA, of treatment with either an anti-dandruff or placebo shampoo.

### DETAILED DESCRIPTION OF THE INVENTION

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

The present invention objectively measures the direct and indirect effects of itchy scalp by employing physiological measurements (electrodermal activity (EDA), heart rate (HR) & respiration rate). EDA, HR and respiration rate data reveal physiological differences between itchy and non itchy respondents.

Psychophysiological Measures

The term “psychophysiological measurement(s)”, as used herein, broadly includes both biological (physiological) measures as well as behavioral measures which measure both the autonomic responses of the subject, as well as learned responses whether executed consciously or sub-consciously, often executed as a learned habit. Specifically, psychophysiology refers to a change in an organism's physiology resulting from a psychological event. Physiological measurements are sometimes referred to as “biometric expressions” or “biometric data.” See e.g., U.S. Pat. No. 5,676,138; U.S. Pat. No. 6,190,314; U.S. Pat. No. 6,309,342; U.S. Pat. No. 7,249,603; and US 2005/0289582. For purposes of clarification, the terms “physiological measurement,” “biometric expression,” “biometric data” “psychophysiological measures” and “psychophysiological data” are used interchangeably herein. Body language, among other things, can non-verbally communicate emotive states via body gestures, postures, body or facial expressions, and the like. Generally, algorithms for physiological measurements can be used to implement embodiments of the present invention. Some embodiments may capture only one or a couple of physiological measurement(s) to reduce costs while other embodiments may capture multiple physiological measurements for more precision. Many techniques have been described in translating physiological measurements or psychophysiological data into an emotional metric data (e.g., type of emotion or emotional levels). See e.g., US 2005/0289582, ¶¶37-44 and the references cited therein. Examples may include Hidden Markov Models, neural networks, and fuzzy logic techniques. See e.g., Comm. ACM, vol. 37, no. 3, pp. 77-84, March 1994. For purposes of clarification, the definition of the term “emotional metric data” subsumes the terms “emotion”, “type of emotion,” and “emotional level.”

Without wishing to be bound by theory, it is generally thought that each emotion can cause a detectable physical response in the body. There are different systems and categorizations of “emotions.” For purposes of this innovation, any set—or even a newly derived set of emotion definitions and hierarchies, can be used which is recognized as capturing at least a human emotion element. For example, refer to Robert Plutchik's defined eight primary emotions of: anger, fear, sadness, joy, disgust, surprise, curiosity, acceptance; or, Paul Ekman's list of basic emotions are: anger, fear, sadness, happiness, disgust. Further well-known is a list by Paul Ekman is his research on facial expressions in humans. Other emotion research focuses on physical displays of emotion including body language of animals and facial expressions in humans.

Generally, autonomic responses and measurements include but are not limited to changes or indications in: body temperature, e.g., measured by conductive or infrared thermometry, facial blood flow, skin impedance, EEG, EKG, blood pressure, blood transit time, heart rate, peripheral blood flow, perspiration or sweat (measured by and non-limiting examples including electrodermal activity (EDA) and galvanic skin response (GSR)), SDNN heart rate variability, pupil dilation, pulmonary data (non-limiting examples including respiration rate, respiratory pace and volume per breath or an average taken,), digestive tract peristalsis, large intestinal motility, and piloerection, i.e., goose bumps or body hair erectile state, saccades, temperature biofeedback, among others. See e.g., US 2007/010066.

In one embodiment, the physiological data comprises cardiac data. Heart Rate (HR) data is one non-limiting example of such psychophysiological data. Cardio vascular monitoring and other cardiac data obtaining techniques are described in US 2003/0149344. A commercial monitor may include the TANITA, 6102 cardio pulse meter. Electro-cardiography, (using a Holter monitor) is another approach. Yet another approach is to employ UWB radar. In another embodiment, ocular physiological data is data obtained from the subject's eye during research. Examples include pupil dilation, blink and eye tracking data.

In general, EDA is the measurement of the resistance to pass current across 2 electrodes on the skin. Psychological or physiological arousal increases skin moisture through sweating and lowers skin resistance. Non-limiting examples of means to measure such resistance are galvanic skin response (GSR), skin conductance resistance (SCR).

Skin conductance, or electrodermal activity, may be measured by the following non-limiting means; galvanic skin response (GSR), electrodermal response (EDR), psychogalvanic reflex (PGR), skin conductance response (SCR), skin conductance level (SCL), skin resistance response (SRR), skin resistance level (SRL), skin potential response (SPR), or skin potential level (SPL). Skin conductance is a method of measuring the electrical conductance of the skin which varies with its moisture level. Conductance is reciprocal to resistance. Without being bound by theory, the sweat glands are controlled by the sympathetic nervous system so skin conductance is used as an indication of psychological or physiological arousal. Therefore, if the sympathetic branch of the autonomic nervous system is highly aroused, then sweat gland activity will also increase, which in turn increases skin conductance. In this way, skin conductance can be used as a measure of emotional and sympathetic responses. See e.g. Electrodermal Activity, Second Edition, by Wolfram Boucsein, Springer-Science+Business Media, LLC 2012.

In an embodiment of the present invention, skin conductance sensors measure the sweat gland activity, which is a sensitive indicator of arousal. Skin conductance is expressed in micro-mho or micro-siemens and increases when the arousal level increases. During relaxation, the skin conductance level normally decreases.

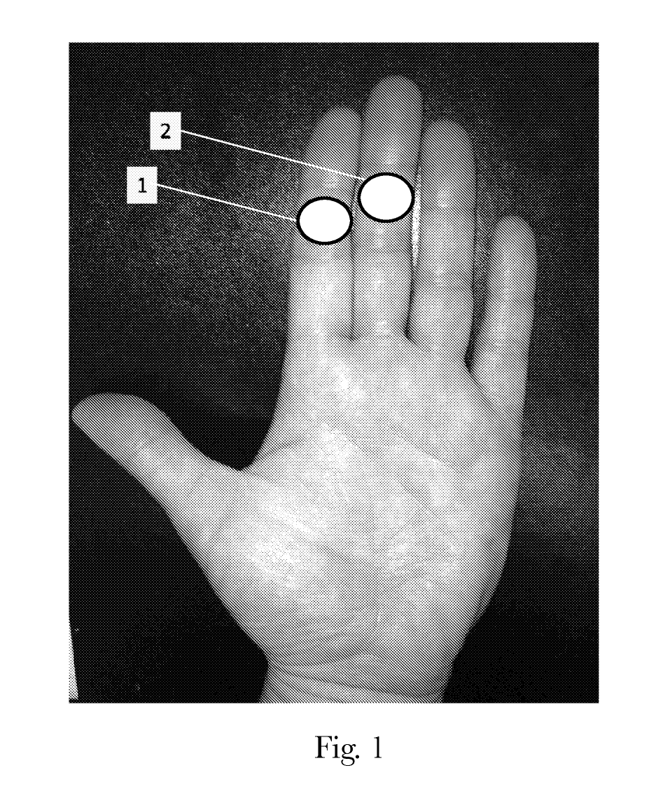

In an embodiment of the present invention, the skin conductance sensor uses finger electrodes. The sensor is designed to measure minute relative changes in skin conductance. FIG. 1 shows a non-limiting example of the placement of a first finger electrode 1 and a second finger electrode 2. Non-limiting examples of such skin conductance sensors include Ag—AgCl electrode types and conductive gel electrodes.

Skin conductance may be recorded using two electrodes, both placed on active sites (bipolar recording). Skin conductance recordings are most commonly taken from locations on the palms of the hands, with several acceptable placements. The most common electrode placements are the thenar eminences of the palms, and the volar surface of the medial or distal phalanges of the fingers. In an embodiment, skin conductance recordings may also be taken from a heel.

Additional physiological measurements can be taken such as: electromyography of the facial, or other muscles; saliva viscosity and volume measures; measurement of salivary amylase activity; body biological function, e.g., metabolism via blood analysis, urine or saliva sample in order to evaluate changes in nervous system-directed responses, e.g., chemical markers can be measured for physiological data relating to levels of neuro-endocrine or endocrine-released hormones; brain function activity. Brain function activity (e.g., location and intensity) may be measured by fMRI (Functional Magnetic Resonance Imaging) or EEG (electroencephalography), forms of medical imaging in this case directed toward the brain. A non-exhaustive list of medical imaging technologies that may be useful for brain function activity understanding, (but can be used for observing other physiological metrics such as the use of ultrasound for heart or lung movement), include fMRI (functional magnetic resonance imaging), MRI magnetic resonance imaging), radiography, fluoroscopy, CT (computated tomography), ultrasonography, nuclear medicine, PET (Positron emission tomography), OT (optical topography), NIRS (near infrared spectroscopy) such as in oximetry, and FNIR (functional near-infrared imaging). In an embodiment, psychophysiological data may include any bodily response associated with psychological or physiological arousal.

Another example of monitoring brain function activity data may include the “brain-machine interface” developed by Hitachi, Inc., measuring brain blood flow. Yet another example includes “NIRS” or near infrared spectroscopy. Yet still another example is electroencephalography (EEG). See also e.g., U.S. Pat. No. 6,572,562.

The term “emotive response indicator(s)” refers to a measure of a physiological or biological process or state of a human or mammal which is believed to be linked or influenced at least in part by the emotive state of the human or mammal at a point or over a period of time. It can also be linked or influenced to just one of the internal feelings at a point or period in time even if multiple internal feelings are present; or, it can be linked to any combination of present feelings. Additionally, the amount of impact or weighting that a given feeling influences an emotive response indicator can vary from person-to-person or other situational factors, e.g., the person is experiencing hunger, to even environmental factors such as room temperature.

The term “emotive state(s)” refers to the collection of internal feelings of the subject at a point or over a period of time. It should be appreciated that multiple feelings can be present such as anxiousness and fear, or anxiousness and delight, among others.

The term “imaging apparatus” is used in the broadest sense and refers to an apparatus for viewing of visual stimulus images including, but not limited to: drawings, animations, computer renderings, photographs, and text, among others. The images can be representations of real physical objects, or virtual images, or artistic graphics or text, and the like. The viewable images can be static, or dynamically changing or transforming such as in sequencing through a deck of static images, showing motions, and the like. The images can be presented or displayed in many different forms including, but not limited to print or painted media such as on paper, posters, displays, walls, floors, canvases, and the like. The images can be presented or displayed via light imaging techniques and displayed for viewing by the subject on a computer monitor, plasma screen, LCD screen, CRT, projection screen, fogscreen, water screen, VR goggles, headworn helmets or eyeglasses with image display screens, or any other structure that allows an image to be displayed, among others. Projected imagery “in air” such as holographic and other techniques are also suitable.

The term “visual stimulus” is used in the broadest sense and refers to any virtual or non-virtual image including but not limited to a product, object, stimulus, and the like, that an individual may view with their eyes. In one embodiment, a non-visual stimulus (e.g., smell, sound, and the like) is substituted for the visual stimulus or is presented concurrently/concomitantly with the visual stimulus. Examples of smells or aromas are described in WO 2007/075205 (pg. 8); U.S. Pat. No. 6,280,751; US 2004/0071757. In one embodiment, the visual stimulus may be archived as a physical image (e.g., photograph) or digital image for analysis or even presentation (such as a report).

In one embodiment, the visual stimulus may be videos which are designed to evoke a strong memory of the context, physiological/kinesthetic experience and the psychological impact of itchy scalp. The videos are able to evoke itch-specific memories/associations (vs. general arousal), since it is these feelings that are relevant to this embodiment. Non-limiting examples of a video stimulus include video depicting eczema, lice and head scratching.

To measure the emotive state of the subject, at least one physiological apparatus is used. For example, the physiological response of a subject's blood pulse can be taken when viewing the visual stimulus. The measured data from the physiological apparatus is synchronized in time with the element to which the viewer has directed their attention at a point in time or over a period of time by computer software. While the recording of clock time is valuable, synchronization does not necessarily need to tag with actual clock time, but associate data with each other that occurred at the same point or interval of time. This allows for later analysis and understanding of the emotive state to various elements. Another aspect of this invention is that certain emotive measurements, e.g., blood pulse measures, can be used to indicate topics or areas, e.g., visual elements, for later research such as a questionnaire if the measurement value(s) meets, exceeds or is less than some pre-determined level set by the researcher.

The physiological apparatus can be worn by the subject, or, it can be a set of fixed sensors or single sensor remotely located from the subject that monitors the physiological responses of the subject when viewing the visual stimulus. For example, the physiological apparatus can be a remotely located infrared camera to monitor changes in body or facial temperature, or the apparatus may be as simple as a watch worn on the wrist of the subject to monitor heart rate. It should be appreciated that in an exemplary embodiment, the physiological apparatus is a wireless physiological apparatus. In other words, the subject is not constricted by any physical wires, e.g., electrical cords, limiting their movement or interaction with the visual stimulus.

The physiological apparatus can further comprise a separate memory device that stores the data obtained from tracking the subject's physiological changes, which may be located on the subject or be remote from the subject. The memory device can then be electronically or wirelessly connected with a separate computer or storage system to transfer the data. The memory device can further comprise a memory disk, cartridge, or other structure to facilitate the ease of transferring data, e.g., flash memory card. The physiological apparatus can also be configured to wirelessly transfer data to a separate data-capturing system that stores the data, e.g., through Bluetooth technology. Either way, the end result is that the data from any eye-tracking apparatus and/or the physiological apparatus is transferred to a separate apparatus that is configured to correlate, evaluate, and/or synchronize both sets of data, among other functions. For purposes of a simplified description, the separate apparatus is described as a data-capturing apparatus. The data-capturing apparatus can be a separate computer, a laptop, a database, server, or any other electronic device configured to correlate, evaluate, and/or synchronize data from the physiological apparatus and/or any eye-tracking apparatus.

The data-capturing apparatus can further comprise additional databases or stored information. For example, known probable emotive states associated with certain physiological or eye-gaze measurement values, or derivative values such as from intermediate analysis, can be stored and looked up in a table within the database and then time-associated, i.e., synchronized, with the viewed element for each or any time interval, or over a period of time, recorded during the period that the subject is viewing the visual stimulus. It should be appreciated that a given physiological measure can also indicate two or more possible responses either singly or in combination. In these cases, all possible responses can be associated with a given time interval in the database.

### Test Methods

Study design

Subjects are instructed on application of a psychophysiological device and escorted to a private area to apply the chest strap and monitor under their clothes against the skin, electrodermal activity electrodes are applied to the fingertips. Psychophysiological measurements begin after device application and continued throughout the duration of the study.

Since itchiness can be transient and not always present even in those subjects who experience scalp itchiness, the subjects are shown a stimulus video to induce itchiness in real time. Subjects are exposed to two different itch videos. In order to create a stable baseline response for the psychophysiological measures all subjects are also exposed to a consistent set of baseline stimuli. These may include but are not limited to, numeracy or literacy working memory tasks (e.g., word search problem) video and or static images with a predicted emotional response (e.g., a picture of a baby commonly results in a positive response, whereas a beautiful beach is calming).

In a non-limiting example, the videos may be designed to evoke a strong memory of the context, physiological/kinesthetic experience and the psychological impact of itchy scalp. The videos may evoke itch-specific memories/associations (vs. general arousal), since it is these feelings that were are most relevant to the project objectives and technology being assessed.

Prior to the study, subjects have a wash-out period with a two weeks use of a commercial shampoo product to remove any previous active, such as an anti-dandruff active, shampoo effects.

Session 1: Subjects have a Baseline psychophysiological assessment, evaluation of response to itch stimulus video/videos. Subjects are then given balanced assignment to a group based on normal product usage and baseline psychophysiological reactivity.

Product Usage: For a usage period, subjects are given either a product containing either an effective technology or a control or placebo treatment.

Session 2: Subjects have a Baseline psychophysiological assessment, and evaluation of response to an itch stimulus video/videos.

Physiological measures are recorded through the session. Analysis is focused on baseline acclimation period, itch stimulus video viewing (Video) and Post-video period.

### EXAMPLES

The following are non-limiting examples.

### Example One

### Evaluation of Itchy and Non-Itchy Subjects

A total of 18 subjects with self-reported itchy scalp and 17 subjects without itchy scalp participate in the study. Upon viewing an itch stimulus video, the psychophysiological attributes of electrodermal activity (EDA), respiration rate and heart rate are acquired. Increased emotive state is represented by an increase in EDA and decreases in respiration and heart rates, the latter due to “fright” element of the so-called “fight or flight” response.”

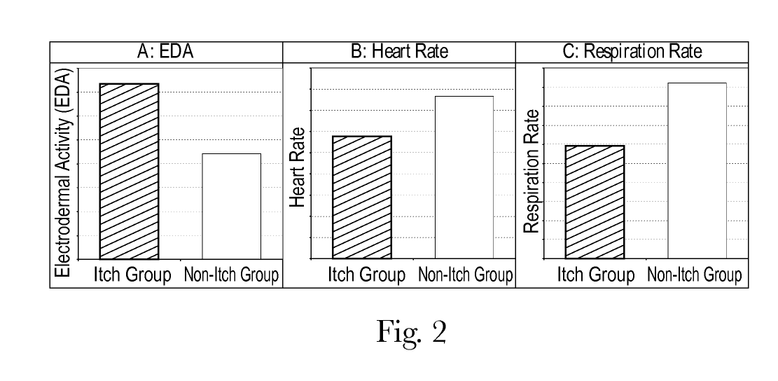

FIGS. 2 A, B and C represent the psychophysiological responses for the self-assessed itch and non-itch groups and show the itchy group demonstrate increased EDA and decreased respiration and heart rates. These results demonstrate that the itchy group is in a more emotionally reactive state upon watching a scalp stimulus video than the non-itchy group and that these psychophysiological responses are relevant noninvasive, objective indications of an individual's state of itch.

### Example Two

### Demonstration of Therapeutic Benefits of Anti-Dandruff Shampoo Amongst Itchy Subjects with a Placebo Control

40 self-assessed scalp itch subjects are divided between two groups, each of which using a shampoo over four weeks. One group uses an anti-dandruff shampoo and the other a placebo shampoo. Psychophysiological measures are recorded at the baseline session as well as after four weeks of product usage, in both cases after watching the stimulus video. All data are normalized using Z-scores.

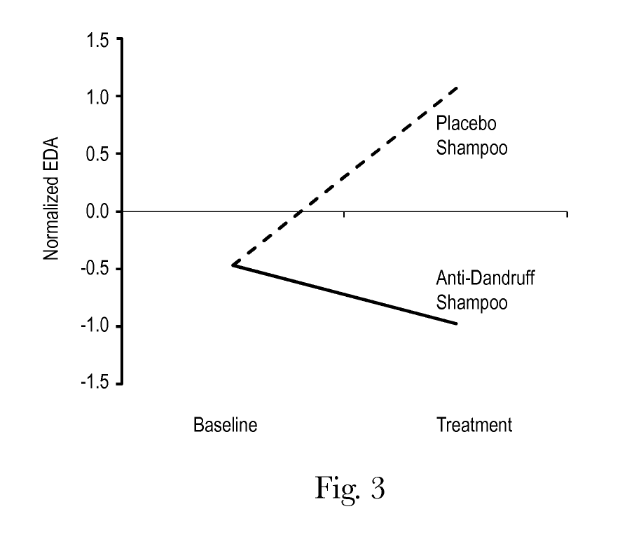

The EDA data is summarized in FIG. 3 and demonstrates that the reduction in itch that accompanies improvement in scalp health due to use of a dandruff treatment shampoo is reflected in a significantly (p=0.05) reduced EDA relative to a placebo shampoo that does not provide the same itch reduction benefit. This demonstrates the capability of EDA to function as an objective, non-invasive measure of itch intensity.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm. ”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) An objective non-invasive method for quantifying itch comprising the steps of:
a) presenting a visual stimulus to a subject, wherein the visual stimulus comprises focusing the subject on itch;
b) collecting psychophysiological data from subject while presenting the visual stimulus;
c) using the psychophysiological data to objectively assess the therapeutic value of a treatment or a product.

2) The method of claim 1 wherein the method further comprises the step of translating an emotive state to an objectively measured psychophysiological data.

3) The method of claim 2 wherein the psychophysiological data is chosen from electrodermal activity, pulmonary data, cardiac data and combinations thereof.

4) method of claim 3 wherein the electrodermal activity is a galvanic skin response (GSR).

5) method of claim 1 wherein the electrodermal activity is collected with a hand, a foot and combinations thereof.

6) method of claim 1 wherein the electrodermal activity is collected with finger of a hand, a palm of a hand, a heel of a foot and combinations thereof.

7) method of claim 6 wherein the electrodermal activity is collected with a first finger electrode and a second finger electrode.

8) method of claim 3 wherein the pulmonary data is a respiration rate.

9) method of claim 3 wherein the cardiac data is a heart rate.

10) method of claim 1 wherein the visual stimulus comprises one or more itch stimuli video.

11) method of claim 10 wherein the itch stimuli video comprises an image selected from the group consisting of eczema, lice, head scratching or combinations thereof.

12) method of claim 4 wherein the electrodermal activity data demonstrates that the reduction in itch that accompanies improvement in scalp health due to use of a dandruff treatment shampoo is significantly reduced electrodermal activity relative to a placebo shampoo.
